(19) United States
(12) Patent Application Publication
(10) Pub. No.: US 2020/0082449 A1
(43) Pub. Date: Mar. 12, 2020

(54) System, Method, and Computer Program for Improving Customer Experience and Sales Effectiveness in the Eye-wear Purchasing Decision

(71) Applicant: EyeOn.pro Ltd., Timnath, CO (US)

(72) Inventors: Craig Dean Hanson, Elk Grove, CA (US); Paul DeVoe, Timnath, CO (US)

(21) Appl. No.: 16/125,010

(22) Filed: Sep. 7, 2018

(51) Int. Cl. G06Q 30/06; G06F 16/9535; G16H 30/20

(52) U.S. Cl. CPC G06Q 30/0621; G16H 30/20; G06F 16/9535

(57) ABSTRACT

The presently disclosed technology include a system, method, and computer program product to causes an optometrist's eyewear inventory to be available in advance of an optometric patient visit. In use, the inventory once only available in the optometry office becomes available, which allows the optometrist to compete with alternative non-optometry eyewear purveyors. Gathering relevant patient preference information, fashion preferences, and personal psychographic information allows the optometry staff to make better eyewear suggestions. The patient can review all eyewear options even when they are not at the optometry office. This process also allows the patient to access virtual or on-line inventory that would not otherwise be easily available to the patient. The patient can share the eyewear selection process with other individuals for assisting in the selection of eyewear. All information is provided to the optometric office with the purpose of optimizing the purchase experience for the specific patient.

### BACKGROUND

Eyewear sales in an optometric practice typically are an important revenue stream to the optometric practice. The practice of frame selection and purchasing by a patient is typically time boxed around the patient's appointment at the optometric office. At that time, the patient may review the available physical inventory, select, and try on frames. Often the purchasing experience is compressed and the patient feels rushed. The optometric patient has a limited opportunity for selection input from family, friends or other stakeholders. When the patient leaves the optometric office without purchasing, it is much more likely they will not purchase eyewear from the that optometric provider. There is a need for improving the purchasing experience giving the patient earlier access to the optometry inventory to promote a positive customer experience and facilitate a purchasing decision.

### FIELD OF THE INVENTION

The present invention relates generally to optimization of purchasing eyewear and the optimization of customer experience in an optometric practice.

### SUMMARY

A system, method, and computer program product are installed which causes the optometrist's eyewear inventory to be available in advance of an optometric patient visit. In use, the inventory once only available in the optometry office becomes available, which allows the optometrist to compete with alternative non-optometry eyewear purveyors. Gathering relevant patient preference information, fashion preferences, and personal psychographic information allows the optometry staff to make better eyewear suggestions. The patient can review all eyewear options even when they are not at the optometry office. This process also allows the patient to access virtual or on-line inventory that would not otherwise be easily available to the patient. The patient can share the eyewear selection process with other individuals for assisting in the selection of eyewear. All information is provided to the optometric office with the purpose of optimizing the purchase experience for the specific patient.

### BRIEF DESCRIPTION OF THE DRAWINGS

FIG. 1 illustrates an exemplary system, in accordance with one embodiment.

FIG. 2 illustrates a network architecture, in accordance with one possible embodiment.

FIG. 3 illustrates a method 300 for enabling optometric patients to review available inventory select eyewear favorites and provide personal information that will enhance the eyewear purchasing experience.

### DETAILED DESCRIPTION

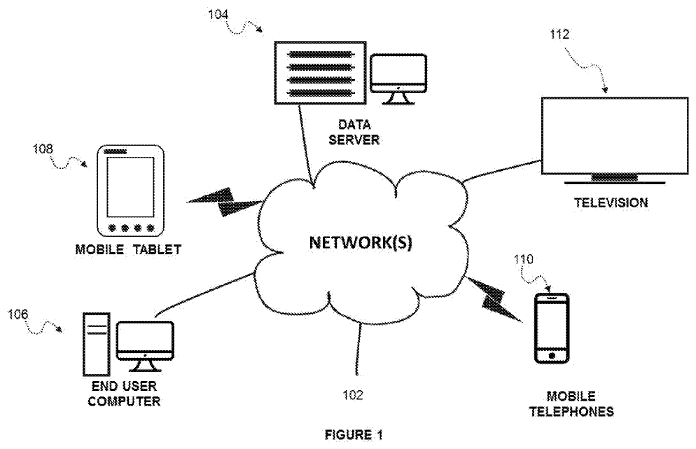

FIG. 1 illustrates a network architecture 100, in accordance with one possible embodiment. As shown, at least one network 102 is provided. In the context of the present network architecture 100, the network 102 may take any form including, but not limited to a telecommunications network, a local area network (LAN), a wireless network, a wide area network (WAN) such as the Internet, peer-to-peer network, cable network, etc. While only one network is shown, two or more similar or different networks 102 may be provided.

Coupled to the network 102 is a plurality of devices. For example, a server computer 104 and an end user computer 106 may be coupled to the network 102 for communication purposes. Such end user computer 106 may include a desktop computer, lap-top computer, and/or any other type of logic. Still yet, various other devices may be coupled to the network 102 including a personal mobile tablet 108, a mobile phone device 110, a television 112, etc.

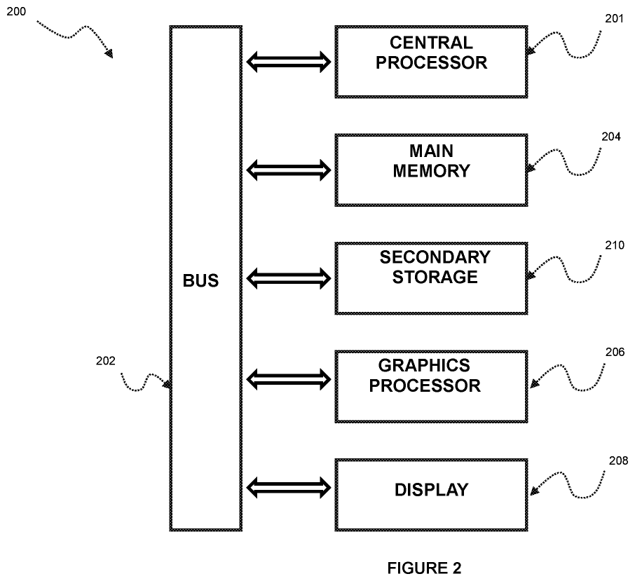

FIG. 2 illustrates an exemplary system 200, in accordance with one embodiment. As an option, the system 200 may be implemented in the context of any of the devices of the network architecture 100 of FIG. 1. Of course, the system 200 may be implemented in any desired environment.

As shown, a system 200 is provided including at least one central processor 201 which is connected to a communication bus 202. The system 200 also includes main memory 204 [e.g. random access memory (RAM), etc.]. The system 200 also includes a graphics processor 206 and a display 208.

The system 200 may also include a secondary storage 210. The secondary storage 210 includes, for example, a hard disk drive and/or a removable storage drive, representing a floppy disk drive, a magnetic tape drive, a compact disk drive, etc. The removable storage drive reads from and/or writes to a removable storage unit in a well-known manner.

Computer programs, or computer control logic algorithms, may be stored in the main memory 204, the secondary storage 210, and/or any other memory, for that matter. Such computer programs, when executed, enable the system 200 to perform various functions (to be set forth below, for example). Memory 204, storage 210 and/or any other storage are possible examples of tangible computer-readable media.

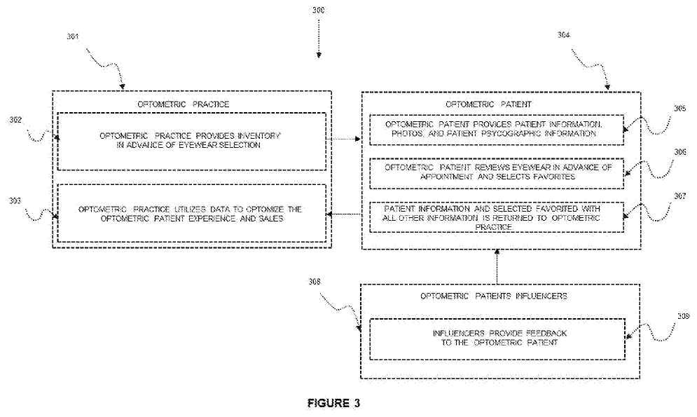

FIG. 3 illustrates a method 300 for enabling optometric patients to review available inventory select eyewear favorites and provide personal information that will enhance the eyewear purchasing experience.

As an option, the method 300 may be carried out in the context of the details of FIGS. 1 and/or 2. Of course, however, the method 300 may be carried out in any desired environment. Further, the aforementioned definitions may equally apply to the description below.

As shown, one or more optometric practice 301 provides access to eyewear inventory in advance of the eyewear selection 302. At least one optometric patient 304 provides patient information, photos, and patient psychographic information. The optometric patient reviews eyewear in advance and selects favorites 306. The optometric patient Influencers 308 are any person or entity that can influence the purchasing decision for the optometric patient. The optometric patients' influencers 309 provide feedback to the optometric patient. Patient information 307 with the patient selected favorites and all other information are stored and returned to the optometric practice. The optometric practice utilizes the data 303 to optimize the optometric patient experience and improve sales conversions.

1. A computer program product embodied on a non-transitory computer readable medium or mobile computing platform, comprising computer code for:
providing patient access to digitally delivered eyewear inventory who have a scheduled appointment with an optometric provider;
digitally storing the current eyewear inventory found in an optometric practice including both physical inventory or virtual inventory;
presenting the optometric practice's current eyewear inventory to patients on a mobile computing or internet-connected computing device;
receiving and storage of patient information, eyewear preferences, eyewear favorites and personal psychographic information;
conveying the patient eyewear preferences to an external mobile computing or internet connected computing device; using the stored information along with the with the patient preferences to improve sales effectiveness.

2. The computer program product of claim 1, wherein the patient causes the inventory to be displayed on a compatible mobile device.

3. The computer program product of claim 1, wherein the patient provides recordable feedback information and preferences about the presented inventory.

4. The computer program product of claim 1, wherein the patient provides feedback, preferences and favorites of the presented inventory.

5. The computer program product of claim 1, wherein the patient provides digital images of themselves.

6. The computer program product of claim 1, wherein the patient provides information regarding their fashion and style preferences and personal psychographic information.

7. The computer program product of claim 1, wherein the patient can share the information, inventory, and images with others with the purpose of assisting the patient with their purchase decision in a social on-line setting.

8. The computer program product of claim 1, wherein the patient information and preferences are used as a basis to recommend alternative eyewear to the patient in the eyewear purchasing decision.

9. The computer program product of claim 1, wherein the patient information and preferences are used as a basis to recommend eyewear to other patients in the eyewear purchasing decision.

10. The computer program product of claim 1, wherein the optometric provider initiates the eyewear purchasing experience in advance by a digital outbound message or by voice call.

11. The computer program product of claim 1, wherein the optometric patient initiates the eyewear purchasing experience in advance by accessing the optometric provider's mobile application, mobile web page, web page, digital advertisement or the provider home on any social platform.

12. The computer program product of claim 1, wherein the optometric provider uses the received and stored information to identify the optometric patient, their preferences and optimize an eye ware purchasing experience.

13. The computer program product of claim 1, wherein the optometric provider is provided literature and material about the patient's inventory preference and favorites to increase patient satisfaction and sales conversion rate.

14. The computer program product of claim 1, wherein advertisements using patient information, fashion preferences, design preferences and personal psychographic information are presented to the optometric patient as the patient is making purchasing decision.

15. A system comprising: a memory system; and one or more processing cores coupled to the memory system and that are each configured for:
providing a patient customized web based application or mobile device application; provides the patient with information gathering interfaces that Identify fashion preferences, design preferences and personal psychographic information;
collects photographs of the patient; presents customers or optometric patients with potential eye ware options;
provides patients a mechanism to identify their preferences or favorites;
provides the optometric professional access to the stored photos, fashion preferences, design preferences and personal psychographic information;
allows optometric practices to optimize customer experience; optometric practices to optimize eye ware frame sales.
